(19) United States
(12) Patent Application Publication
(10) Pub. No.: US 2008/0161916 A1
(43) Pub. Date: Jul. 3, 2008

(54) BREAST PROSTHESIS/ENHANCEMENT COVER

(76) Inventor: Virginia V. Heitman, Naples, FL (US)

Correspondence Address: EDWARD M. LIVINGSTON, PA, 963 TRAIL TERRACE DRIVE, NAPLES, FL, 34103, US

(21) Appl. No.: 11/618,250

(22) Filed: Dec. 29, 2006

(51) Int. Cl. A61F 2/52; A41C 3/10

(52) U.S. Cl. 623/7; 450/57

(57) ABSTRACT

Abreast prosthesis/enhancement cover (1) having a perimeter (6), a first rear section (7) secured to a portion of the front section perimeter (6) and a second rear section (8) secured to a portion of the front section perimeter (6) in such a manner so as the second rear section (8) overlaps a portion of the first rear section (7) to form a pocket (11) for the acceptance of a breast prosthesis/enhancement (3). A securing means (4) is located on the front section (2), which is preferably an adhesive strip of a predetermined size. The sections (2), (7) and (8) of the cover (1) are preferably made of a soft, non-irritating material so as to be comfortable to the user. An optional faux nipple (5) may also be located on the front section (2) so as to provide a natural appearance when the cover (1) is used.

### BACKGROUND OF THE INVENTION

This invention relates to breast prostheses and breast enhancements, more particularly, a breast prosthesis or breast enhancement cover that enables a woman to quickly and easily wear a breast prosthesis or breast enhancement without a support garment and in a sanitary manner.

Many women, particularly breast cancer survivors who have undergone a mastectomy, use breast prostheses, also known as breast forms, in order to gain emotional and physical confidence by having a more traditional womanly shape. The breast prosthesis is made of a specialized material, such as silicone gel, fiberfill, or polyurethane foam, so as to have a natural shape and feel. Usually the breast prosthesis is positioned within a support garment having straps, such as a mastectomy bra, which has at least one pocket that is sized and shaped to accommodate a breast prosthesis.

However, because a mastectomy bra must be worn in order to wear such breast prostheses, women refrain from wearing tank tops, spaghetti straps and strapless shirts and dresses as the mastectomy bra straps are visible. Although women may wear a form-fitting garment, such as knit tops having a built-in or “shelf” bra or a stretch-type camisole foundation, wherein a prosthesis may be slipped-into and held against the body by the shelf bra, the prosthesis will usually slide around within the built-in bra or foundation as the prosthesis is not secured to the built-in bra or foundation, thereby resulting in an unnatural look. In addition, because the prosthesis is positioned directly against the skin, sweat, bacteria and other microorganisms accumulate on the prosthesis, thereby requiring the user to continuously wash the prosthesis, a tedious process which is described below.

Furthermore and as with any other bra, it is often more comfortable for a woman to relax around the house and go to sleep without wearing a bra. However, if the woman feels more confident physically and emotionally when wearing a breast prosthesis, she is faced with the difficult decision of wearing a mastectomy bra with exposed bra straps when wearing a nightgown or lingerie at night or wearing no breast prosthesis at all.

An alternative means for wearing a breast prosthesis or breast enhancement is by using a cover in conjunction with the prosthesis/enhancement wherein the cover includes a tab. To use, a woman inserts the prosthesis/enhancement within the cover and secures the cover to a strap of a traditional bra. However, as discussed above, a support means, such as a bra, must still be worn in conjunction with the cover wherein straps are visible, an option which is not always viable.

One way in which women have worn breast prostheses or breast enhancements without the use of a support garment is by securing the tab of a breast prosthesis and/or breast enhancement cover directly to the article of clothing itself. However, because a pin must puncture through the article of clothing, the pin itself is visible on the outside of the article of clothing, thereby making it noticeable that the wearer is wearing a prosthesis or enhancement. In addition, puncture holes may remain in the article of clothing.

Furthermore, as breast prostheses are expensive, women usually own a single prosthesis or one pair as in the case of women who have undergone a double mastectomy. The wearer usually wears the prosthesis on a daily basis so as to have a uniform, natural look. Therefore, as the same breast prosthesis is worn on a daily basis, it is important to keep the prosthesis in a clean and sanitary condition as it is not cost efficient to purchase many prostheses.

However, as the prosthesis is not typically an item that is machine washable, the user must take the time to hand wash the prosthesis and allow the prosthesis to air dry. This process is very time consuming and, as it takes an extended amount of time to dry the prosthesis, washing it may prevent the wearer from going out of the home until the prosthesis is dry as she is unable to wear a damp prosthesis.

Moreover, these problems are not only related to breast prostheses, but are also related to breast enhancers which are typically used when a woman has undergone a partial mastectomy or any women simply desiring to increase the size appearance of her breasts.

Thus, there exists the need for a breast prosthesis and breast enhancement cover that enables a woman to quickly and easily wear a breast prosthesis or breast enhancement without a support garment and in a sanitary manner.

The relevant prior art includes the following patents:

| Patent No. | | |
|---|---|---|
| (U.S. unless stated otherwise) | Inventor | Issue/Publication Date |
| 5,066,302 | Rice | Nov. 19, 1991 |
| 4,369,792 | Miller | Jan. 25, 1983 |
| 4,100,621 | Ettipio | Jul. 18, 1978 |
| 2,875,449 | Morris | Mar. 03, 1959 |
| 3,807,412 | Connelly | Apr. 30, 1974 |
| 7,056,186 | Weyenberg et al. | Jun. 06, 2006 |
| ZA 20000523A | Merry | Jun. 27, 2001 |

### SUMMARY OF THE INVENTION

The primary object of the present invention is to provide a breast prosthesis/enhancement cover that permits a user to wear a breast prosthesis or breast enhancement in a sanitary manner.

A further object of the present invention is to provide a breast prosthesis/enhancement cover that permits a user to wear a breast prosthesis or breast enhancement without a bra or other support garment.

An even further object of the present invention is to provide a breast prosthesis/enhancement cover that is disposable.

A further object of the present invention is to provide a breast prosthesis/enhancement cover that is securable to an article of clothing.

An even further object of the present invention is to provide a breast prosthesis/enhancement cover that does not damage an article of clothing.

An even further object of the present invention is to provide a breast prosthesis/enhancement cover that is easy to use.

The present invention fulfills the above and other objects by providing a breast prosthesis/enhancement cover having a front section having a perimeter that forms a predetermined shape, a first rear section secured to a portion of the perimeter and a second rear section secured to a portion of the perimeter in such a manner so as the second rear section overlaps a portion of the first rear section to form a pocket. A securing means located on the front section, which is preferably an adhesive strip of a predetermined size. The sections of the cover are preferably made of a soft, non-irritating material so as to be comfortable to the user. In addition, the front section and rear sections may include a predetermined amount of cushion. Finally, an optional faux nipple may also be located on the front section so as to provide a natural appearance when the cover is used.

To use the present invention, a user inserts a breast prosthesis or breast enhancement within the cover via the pocket such that the front of the breast prosthesis or breast enhancement is adjacent to the front section. The user then manipulates the cover so as to properly encase the breast prosthesis or enhancement. Finally, the user removes a protective strip located on the front section to expose an adhesive strip, positions the cover in a desired location on an interior of an article of clothing and presses the adhesive strip to the clothing so as to adhere the cover, and breast prosthesis/enhancement located therein, to the clothing. As the breast prosthesis/enhancement is not placed within a mastectomy bra and the cover is directly securable to the article of clothing without a pin, a user may wear any article of clothing, such as tank tops, spaghetti straps, strapless shirts and dresses, nightgowns and lingerie, that are form fitting, including but not limited to garments made with Lycra®. When the user no longer desires to wear the prosthesis, the user detaches the cover from the clothing, removes the prosthesis from the cover and simply disposes of the cover. Thus, a person may use a new cover each time she desires to wear a prosthesis, thereby promoting sanitary conditions of the prosthesis.

The above and other objects, features and advantages of the present invention should become even more readily apparent to those skilled in the art upon a reading of the following detailed description in conjunction with the drawings wherein there is shown and described illustrative embodiments of the invention.

### BRIEF DESCRIPTION OF THE DRAWINGS

In the following detailed description, reference will be made to the attached drawings in which:

FIG. 1 is a front plan view of a breast prosthesis/enhancement cover of the present invention housing a breast prosthesis;

FIG. 2 is a plan rear view of the embodiment of FIG. 1;

FIG. 3 is a side view of the embodiment of FIG. 1;

FIG. 4 is a side view of the breast prosthesis/enhancement cover of the present invention; and

FIG. 5 is a side view of the breast prosthesis/enhancement cover in use.

### DESCRIPTION OF THE PREFERRED EMBODIMENTS

For purposes of describing the preferred embodiment, the terminology used in reference to the numbered components in the drawings is as follows:

| | |
|---|---|
| 1. | breast |
| | prosthesis/enhancement |
| | cover, generally |
| 2. | front section |
| 3. | breast |
| | prosthesis/enhancement |
| 4. | securing means |
| 5. | faux nipple |
| 6. | front section perimeter |
| 7. | first rear section |
| 8. | second rear section |
| 9. | overlap |
| 10. | cushion |
| 11. | pocket |
| 12. | clothing |
| 13. | chest |
| 14. | perimeter |
| 15. | pocket access |
| 16. | exterior |
| 17. | interior |

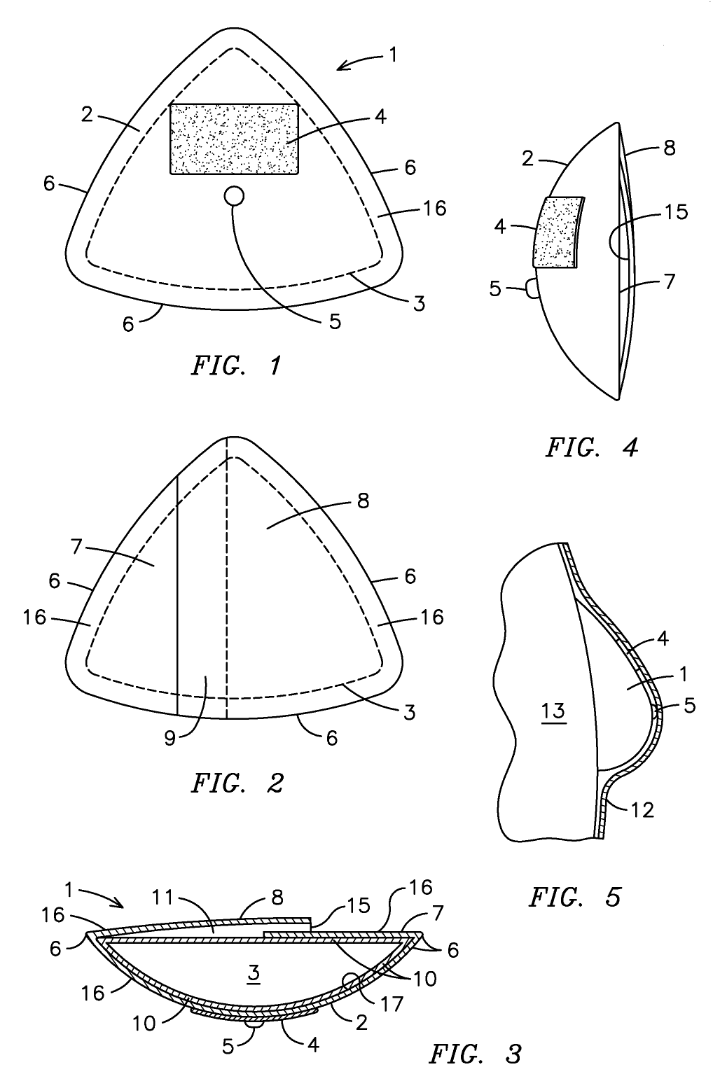

With reference to FIG. 1, a front plan view of a breast prosthesis/enhancement cover of the present invention housing a breast prosthesis is shown. The breast prosthesis/enhancement cover, generally 1, includes a front section 2 having a predetermined front section perimeter 6, which may be circular, triangular, rectangular or any other shape to form a breast prosthesis/enhancement cover 1 of a predetermined shape and size. A securing means 4, which is preferably at least one strip of adhesive material, is located on an exterior 16 of the front section 2 of the breast prosthesis/enhancement cover 1. However, other securing means 4, such as hook and loop type fastening material, may also be used. An optional faux nipple 5 may be located on the front section 2 of the breast prosthesis/enhancement cover 1. A breast prosthesis/enhancement 3 is housed within the breast prosthesis/enhancement cover 1 when in use.

Next, FIG. 2 shows a plan rear view of the embodiment of FIG. 1. A first rear section 7 having an exterior 16 is secured to the front section 2 along a portion of the front section perimeter 6. A second rear section 8 having an exterior 16 is secured to the front section 2 along a portion of the front section perimeter 6 in such a manner to form an overlap 9 as a portion of the second rear section 8 overlaps a portion of the first rear section 7.

In FIG. 3, a side view of the embodiment of FIG. 1 is shown. The breast prosthesis/enhancement 3 is housed within a pocket 11 of the breast prosthesis/enhancement cover 1 when in use so as to be completely encased within the breast prosthesis/enhancement cover 1. An optional cushion 10 may be secured to an interior 17 of the front section 2 and to an interior 17 of the rear sections 7 and 8 of the breast prosthesis/enhancement cover 1 to provide additional comfort to the wearer. To insert a breast prosthesis/enhancement 3 within a breast prosthesis/enhancement cover 1 of the present invention, a person first inserts the breast prosthesis/enhancement 3 into the pocket access 15 formed by the first rear section 7 and second rear section 8. Then, the user manipulates the first rear section 7 so as to place the breast prosthesis/enhancement 3 within the pocket 11 that is created by the first rear section 7 and the second rear section 8.

FIG. 4 shows a side view of the breast prosthesis/enhancement cover of the present invention. Because the second rear section 8 is secured over the first rear section 7, a pocket access 15 is formed.

Finally, FIG. 5 shows a side view of the breast prosthesis/enhancement cover in use. Once the breast prosthesis/enhancement 3 is located within the pocket 11 of the breast prosthesis/enhancement cover 1, a user positions the breast prosthesis/enhancement cover 1 to a desired location on a chest 13 and secures the breast prosthesis/enhancement cover 1 to an interior of a piece of clothing 12 via a securing means 4, which is preferably at least one adhesive strip.

Because the breast prosthesis/enhancement 3 is encased within the breast prosthesis/enhancement cover 1, the breast prosthesis/enhancement 3 is kept in a sanitary condition because is it not in direct contact with the user's skin. In addition, the breast prosthesis/enhancement cover 1 may be disposable, thereby permitting a user to use a clean breast prosthesis/enhancement cover 1 on a daily basis.

Furthermore, because a securing means 4 that does not involve a pin is used to secure the breast prosthesis/enhancement cover 1 to the clothing 12, a wearer is not limited in the types of clothing 12 she wears as she need not wear a support garment or bra to use the present invention. In addition, she need not be concerned with a portion of a pin being visible or hole marks being left in the clothing 12 as is caused when using pins.

Therefore, the use of the present invention permit a person to quickly and easily wear a breast prosthesis and/or breast enhancement without a support garment and in a sanitary manner.

It is to be understood that while a certain form of the invention is illustrated, it is not to be limited to the specific form or arrangement of parts herein described and shown. It will be apparent to those skilled in the art that various changes may be made without departing from the scope of the invention and the invention is not be considered limited to what is shown and described in the specification and drawings.

Having thus described my invention, I claim:

1. A breast prosthesis/enhancement cover comprising:
a front section having a perimeter that forms a predetermined shape;
said front section having an exterior and an interior;
a first rear section secured along a portion of the front section perimeter;
said first rear section having an exterior and an interior;
a second rear section secured along a portion of the front section perimeter wherein a portion of the second rear section overlaps a portion of the first rear section to form a pocket access;
said second rear section having an exterior and an interior;
a pocket located between the front section, first rear section and second rear section for the acceptance of a breast prosthesis/enhancement; and
a securing means located on the front section exterior.

2. The breast prosthesis/enhancement cover of claim 1 wherein:
said securing means is at least one adhesive strip.

3. The breast prosthesis/enhancement cover of claim 1 wherein:
said securing means is hook and loop fastening material.

4. The breast prosthesis/enhancement cover of claim 1 further comprising:
a faux nipple located on the front section exterior.

5. The breast prosthesis/enhancement cover of claim 2 further comprising:
a faux nipple located on the front section exterior.

6. The breast prosthesis/enhancement cover of claim 3 further comprising:
a faux nipple located on the front section exterior.

7. The breast prosthesis/enhancement cover of claim 1 further comprising:
a cushion located on the front section interior.

8. The breast prosthesis/enhancement cover of claim 2 further comprising:
a cushion located on the front section interior.

9. The breast prosthesis/enhancement cover of claim 3 further comprising:
a cushion located on the front section interior.

10. The breast prosthesis/enhancement cover of claim 4 further comprising:
a cushion located on the front section interior.

11. The breast prosthesis/enhancement cover of claim 5 further comprising:
a cushion located on the front section interior.

12. The breast prosthesis/enhancement cover of claim 6 further comprising:
a cushion located on the front section interior.

13. The breast prosthesis/enhancement cover of claim 1 further comprising:
a cushion located on the first rear section interior; and
a cushion located on the second rear section interior.

14. The breast prosthesis/enhancement cover of claim 2 further comprising:
a cushion located on the first rear section interior; and
a cushion located on the second rear section interior.

15. The breast prosthesis/enhancement cover of claim 3 further comprising:
a cushion located on the first rear section interior; and
a cushion located on the second rear section interior.

16. A breast prosthesis/enhancement cover comprising:
a front section having a perimeter that forms a predetermined shape;
said front section having an exterior and an interior;
a first rear section secured along a portion of the first edge and along a portion of the third edge of the front section;
said first rear section having an exterior and an interior;
a second rear section secured along a portion of the first edge, along a portion of the second edge and along a portion of the third edge wherein a portion of the second rear section overlaps a portion of the first rear section to form a pocket access;
said second rear section having an exterior and an interior;
a pocket located between the front section, first rear section and second rear section for the acceptance of a breast prosthesis/enhancement; and
at least one adhesive strip located on the front section exterior.

17. The breast prosthesis/enhancement cover of claim 16 further comprising:
a cushion located on the front section interior.

18. The breast prosthesis/enhancement cover of claim 16 further comprising:
a faux nipple located on the front section exterior.

19. The breast prosthesis/enhancement cover of claim 17 further comprising:
a faux nipple located on the front section exterior.

20. A breast prosthesis/enhancement cover comprising:
a front section having a perimeter that forms a predetermined shape;
said front section having an exterior and an interior;
a first rear section secured along a portion of the first edge and along a portion of the third edge of the front section;
said first rear section having an exterior and an interior;
a second rear section secured along a portion of the first edge, along a portion of the second edge and along a portion of the third edge wherein a portion of the second rear section overlaps a portion of the first rear section to form a pocket access;
said second rear section having an exterior and an interior;
a pocket located between the front section, first rear section and second rear section for the acceptance of a breast prosthesis/enhancement;
at least one adhesive strip located on the front section exterior; and
a faux nipple located on the front cover exterior.
